(19) United States
(12) Patent Application Publication
(10) Pub. No.: US 2026/0023066 A1
(43) Pub. Date: Jan. 22, 2026

(54) SYSTEMS AND METHODS FOR TESTING HYDROCARBON EMULSIONS

(71) Applicant: Saudi Arabian Oil Company, Dhahran (SA)

(72) Inventor: Maher Maqbool Shariff, Dhahran (SA)

(21) Appl. No.: 18/779,876

(22) Filed: Jul. 22, 2024

(51) Int. Cl. G01N 33/28

(52) U.S. Cl. CPC G01N 33/2823

(57) ABSTRACT

A hydrocarbon emulsion testing process includes feeding multiple hydrocarbon emulsion samples into a hydrocarbon emulsion testing system. At least one sample is heated in a heating unit. At least one sample is injected with a demulsifier. Each of the samples if separated into an oil phase, a water phase, and an emulsion phase. The separated volumes of at least one of the respective oil phases, water phases, and emulsion phase are compared to determine a terminal temperature of the hydrocarbon emulsion testing system.

### TECHNICAL FIELD

This disclosure relates to systems and methods for testing hydrocarbon emulsions, and more particularly, systems and methods for testing hydrocarbon emulsions through multiple emulsion separation techniques that include at least one of heat or demulsifiers.

### BACKGROUND

Hydrocarbon emulsions typically include a mixed-phase fluid that includes oil, gas, and water. Often, hydrocarbon emulsions are formed inside multiphase oil, water, and gas separation vessels at a gas oil separation plant (GOSP). Hydrocarbon emulsions are considered to be “tight” when it is difficult to separate the different phases within the emulsion.

### SUMMARY

In an example implementation, a hydrocarbon emulsion testing process includes feeding a first sample of a hydrocarbon emulsion at a first temperature into a hydrocarbon emulsion testing system (HETS). The hydrocarbon emulsion includes an oil phase, a water phase, and an emulsion phase. The hydrocarbon emulsion testing process includes separating, in a separation tank of the HETS, the first sample of the hydrocarbon emulsion at the first temperature into a first volume of the oil phase, a first volume of the water phase, and a first volume of an emulsion phase; feeding a second sample of the hydrocarbon emulsion at the first temperature into the HETS; heating, in a heating unit of the HETS, the second sample of the hydrocarbon emulsion from the first temperature to a second temperature; separating, in the separation tank, the second sample of the hydrocarbon emulsion at the second temperature into a second volume of the oil phase, a second volume of the water phase, and a second volume of the emulsion phase; feeding a third sample of the hydrocarbon emulsion at the first temperature into the HETS injecting, from a demulsifier tank of the HETS, a demulsifier into the third sample of the hydrocarbon emulsion; heating, in the heating unit, the third sample of the hydrocarbon emulsion from the first temperature to a third temperature; separating, in the separation tank, the third sample of the hydrocarbon emulsion at the third temperature into a third volume of the oil phase, a third volume of the water phase, and a third volume of the emulsion phase; comparing the first, second, and third volumes of at least one of the oil phase, the water phase, or the emulsion phase; and based on the comparison, determining a terminal temperature of the HETS.

An aspect combinable with the example implementation includes, based on at least one of the comparison or the determined terminal temperature, determining an optimum volume of the injected demulsifier.

In another aspect combinable with one, some, or all of the previous aspects, comparing the first, second, and third volumes of at least one of the oil phase, the water phase, or the emulsion phase includes comparing the first, second, and third volumes of the oil phase; and comparing the first, second, and third volumes of the water phase.

In another aspect combinable with one, some, or all of the previous aspects, determining the terminal temperature of the HETS includes at least one of: determining that the third volume of the oil phase is greater than the first and second volumes of the oil phase and is a maximum volume of the oil phase within the hydrocarbon emulsion; or determining that the third volume of the water phase is greater than the first and second volumes of the water phase and is a maximum volume of the water phase within the hydrocarbon emulsion.

In another aspect combinable with one, some, or all of the previous aspects, determining the terminal temperature of the HETS includes determining that the third volume of the oil phase is greater than the first and second volumes of the oil phase and is a maximum volume of the oil phase within the hydrocarbon emulsion; and determining that the third volume of the water phase is greater than the first and second volumes of the water phase and is a maximum volume of the water phase within the hydrocarbon emulsion.

In another aspect combinable with one, some, or all of the previous aspects, heating the second sample of the hydrocarbon emulsion and the third sample of the hydrocarbon emulsion in the heating unit of the HETS includes heating, in a fluid-to-fluid heat exchanger of the heating unit, the second sample of the hydrocarbon emulsion with a heating fluid that includes heat energy from solar energy; and heating, in the fluid-to-fluid heat exchanger, the third sample of the hydrocarbon emulsion with the heating fluid that includes heat energy from solar energy.

In another aspect combinable with one, some, or all of the previous aspects, heating the second sample of the hydrocarbon emulsion and the third sample of the hydrocarbon emulsion in the heating unit of the HETS includes heating, in an electric heater of the heating unit, the second sample of the hydrocarbon emulsion with heat energy from solar energy; and heating, in the electric heater, the third sample of the hydrocarbon emulsion with heat energy from solar energy.

In another aspect combinable with one, some, or all of the previous aspects, each of the first, second, and third samples of the hydrocarbon emulsion is exclusive of demulsifier prior to the feeding into the HETS.

In another aspect combinable with one, some, or all of the previous aspects, the demulsifier includes a first demulsifier, and the process includes feeding a fourth sample of the hydrocarbon emulsion at the first temperature into the HETS; injecting, from the demulsifier tank, a second demulsifier into the fourth sample of the hydrocarbon emulsion, the second demulsifier different than the first demulsifier; heating, in the heating unit, the fourth sample of the hydrocarbon emulsion from the first temperature to the third temperature; separating, in the separation tank, the fourth sample of the hydrocarbon emulsion at the third temperature into a fourth volume of the oil phase, a fourth volume of the water phase, and a fourth volume of the emulsion phase; comparing the third and fourth volumes of at least one of the oil phase, the water phase, or the emulsion phase; and based on the comparison, selecting one of the first or second demulsifiers as an optimum demulsifier.

In another aspect combinable with one, some, or all of the previous aspects, the terminal temperature is the third temperature.

In another aspect combinable with one, some, or all of the previous aspects, wherein heating the third sample of the hydrocarbon emulsion from the first temperature to the third temperature include heating, in the heating unit, the third sample of the hydrocarbon emulsion that includes the demulsifier from the first temperature to the third temperature.

In another example implementation, a hydrocarbon emulsion testing system includes a fluid feeding system including at least one pump and a fluid piping network. The fluid feeding system is configured to feed a first sample of a hydrocarbon emulsion at a first temperature into the fluid piping network, the hydrocarbon emulsion including an oil phase, a water phase, and an emulsion phase; feed a second sample of the hydrocarbon emulsion at the first temperature into the fluid piping network; feed a third sample of the hydrocarbon emulsion at the first temperature into the fluid piping network. The hydrocarbon emulsion testing system includes a demulsifier tank fluidly coupled within the fluid piping network and configured to inject a demulsifier into the third sample of the hydrocarbon emulsion. The hydrocarbon emulsion testing system includes a heating unit thermally coupled to the fluid piping network and configured to heat the second sample of the hydrocarbon emulsion from the first temperature to a second temperature; and heat the third sample of the hydrocarbon emulsion from the first temperature to a third temperature. The hydrocarbon emulsion testing system includes a separation tank fluidly coupled within the fluid piping network and configured to separate the first sample of the hydrocarbon emulsion at the first temperature into a first volume of the oil phase, a first volume of the water phase, and a first volume of an emulsion phase; separate the second sample of the hydrocarbon emulsion at the second temperature into a second volume of the oil phase, a second volume of the water phase, and a second volume of the emulsion phase; and separate the third sample of the hydrocarbon emulsion at the third temperature into a third volume of the oil phase, a third volume of the water phase, and a third volume of the emulsion phase. The hydrocarbon emulsion testing system includes a control system configured to perform operations that include comparing the first, second, and third volumes of at least one of the oil phase, the water phase, or the emulsion phase; and based on the comparison, determining a terminal temperature.

In an aspect combinable with the example implementation, the operations include determining an optimum volume of the injected demulsifier based on at least one of the comparison or the determined terminal temperature.

In another aspect combinable with one, some, or all of the previous aspects, the operation of comparing the first, second, and third volumes of at least one of the oil phase, the water phase, or the emulsion phase includes comparing the first, second, and third volumes of the oil phase; and comparing the first, second, and third volumes of the water phase.

In another aspect combinable with one, some, or all of the previous aspects, the operation of determining the terminal temperature of the HETS includes at least one of determining that the third volume of the oil phase is greater than the first and second volumes of the oil phase and is a maximum volume of the oil phase within the hydrocarbon emulsion; or determining that the third volume of the water phase is greater than the first and second volumes of the water phase and is a maximum volume of the water phase within the hydrocarbon emulsion.

In another aspect combinable with one, some, or all of the previous aspects, the operation of determining the terminal temperature of the HETS includes determining that the third volume of the oil phase is greater than the first and second volumes of the oil phase and is a maximum volume of the oil phase within the hydrocarbon emulsion; and determining that the third volume of the water phase is greater than the first and second volumes of the water phase and is a maximum volume of the water phase within the hydrocarbon emulsion.

In another aspect combinable with one, some, or all of the previous aspects, the heating unit includes a fluid-to-fluid heat exchanger configured to heat the second sample of the hydrocarbon emulsion with a heating fluid that includes heat energy from solar energy; and heat the third sample of the hydrocarbon emulsion with the heating fluid that includes heat energy from solar energy.

In another aspect combinable with one, some, or all of the previous aspects, the heating unit includes an electric heater configured to heat, the second sample of the hydrocarbon emulsion with heat energy from solar energy; and heat the third sample of the hydrocarbon emulsion with heat energy from solar energy.

In another aspect combinable with one, some, or all of the previous aspects, each of the first, second, and third samples of the hydrocarbon emulsion is exclusive of demulsifier prior to the feeding into the HETS.

In another aspect combinable with one, some, or all of the previous aspects, the demulsifier includes a first demulsifier.

In another aspect combinable with one, some, or all of the previous aspects, the fluid feeding system is configured to feed a fourth sample of the hydrocarbon emulsion at the first temperature into the fluid piping network.

In another aspect combinable with one, some, or all of the previous aspects, the demulsifier tank is configured to inject a second demulsifier into the fourth sample of the hydrocarbon emulsion.

In another aspect combinable with one, some, or all of the previous aspects, the heating unit is configured to heat the fourth sample of the hydrocarbon emulsion from the first temperature to a fourth temperature.

In another aspect combinable with one, some, or all of the previous aspects, the separation tank is configured to separate the fourth sample of the hydrocarbon emulsion at the fourth temperature into a fourth volume of the oil phase, a fourth volume of the water phase, and a fourth volume of an emulsion phase.

In another aspect combinable with one, some, or all of the previous aspects, the operations include comparing the third and fourth volumes of at least one of the oil phase, the water phase, or the emulsion phase; and based on the comparison, selecting one of the first or second demulsifiers as an optimum demulsifier.

In another aspect combinable with one, some, or all of the previous aspects, the terminal temperature is the third temperature.

In another aspect combinable with one, some, or all of the previous aspects, the heating unit is positioned downstream of the demulsifier tank in the fluid piping network, and the heating unit is configured to heat a mixture of the third sample of the hydrocarbon emulsion and the demulsifier from the first temperature to the third temperature.

Implementations of hydrocarbon emulsion testing systems and methods according to the present disclosure may include one or more of the following features. For example, implementations according to the present disclosure can provide for enhanced separation of water from tight emulsion in particular oil field processing facilities. In another example, implementations according to the present disclosure can provide for cost effective testing of demulsifiers while providing effective results. Further, implementations according to the present disclosure can provide for enhanced oil-water separation in while minimizing demulsifier costs and usage.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a schematic diagram of an example implementation of a hydrocarbon emulsion testing system according to the present disclosure.

FIG. 2 is a schematic diagram of a multiphase fluid production vessel according to the present disclosure.

FIG. 3 is a schematic diagram of another example implementation of a hydrocarbon emulsion testing system according to the present disclosure.

FIG. 4 is a schematic illustration of an example controller (or control system) for a hydrocarbon emulsion testing system according to the present disclosure.

### DETAILED DESCRIPTION

The present disclosure describes example implementations of a hydrocarbon emulsion testing system (HETS) that can be operated to separate tight emulsions that can, for example, form inside multiphase oil, water, and gas separation vessels at a gas oil separation plants (GOSP). In example aspects, emulsion samples are collected from within a pressurized gravity separation vessels, such as a high pressure production trap (HPPT) or low pressure production trap (LPPT) (for example, onsite at the GOSP). The samples collected can be fed into a HETS for testing under different and varying physical conditions. For example, the HETS can use heat one or more samples to break tight emulsion layers formed inside the separation vessels by increasing its temperature incrementally in a batch mode. As another example, the HETS can use heat coupled with demulsifier injection to break tight emulsion layers. Thus, in some aspects, example implementations of a HETS can separate tight emulsions in a sequential batch mode process that combines heating and demulsifier injection in sequence. As such, example implementations of the HETS according to the present disclosure can help resolve or remedy the problem of the formation of tight emulsions at some GOSPs, which can be an adverse hindrance that needs to be mitigated.

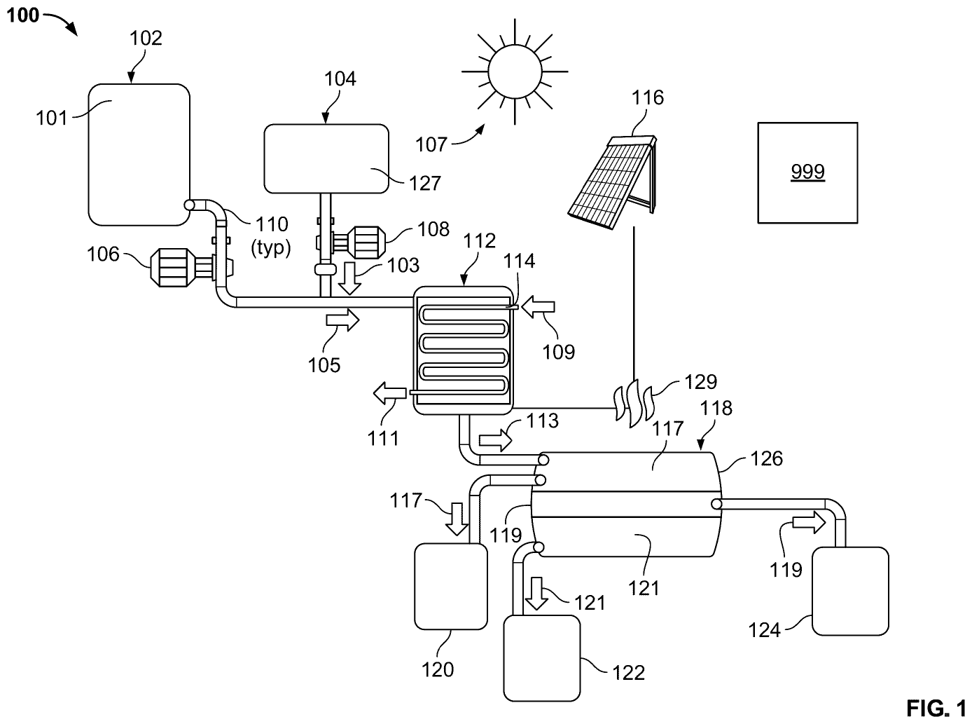

FIG. 1 is a schematic diagram of an example implementation of a hydrocarbon emulsion testing system (HETS) 100 according to the present disclosure. In some aspects, the HETS 100 can be a stand along system as part of an independent laboratory remote from a GOSP. Alternatively, the HETS 100 can be implemented as part of, and adjacent or within, a GOSP.

In this example, the HETS 100 includes an emulsion tank 102 in which an emulsion 101 (for example, a multiphase fluid that includes oil, water, emulsion, gas or a combination thereof) is enclosed. The emulsion tank 102 is fluidly coupled within the HETS 100, as are the other described components, with piping conduits 110 (for example, metallic, non-metallic). As shown here, a pump 106 is fluidly coupled within the piping conduits 110 to circulate the emulsion 101 within the HETS 100 (or at least a portion thereof).

In this example, a demulsifier tank 104 that encloses a demulsifier 127 is fluidly coupled within the HETS 100 through piping conduits 110. The demulsifier 127, in some aspects, is a chemical compound formulated to break emulsion layers within the emulsion 101 (and that typically form inside of oil and water production vessels). Demulsifier 127 can be injected into the HETS 100 and in contact with the emulsion 101 through operation of pump 108. Thus, in some example operations (as described in more detail herein), a mixed emulsion-demulsifier fluid 105 is circulated to a heating unit 112. In some example operations (as described in more detail herein), however, the fluid 105 is the emulsion 101 (in other words, with no demulsifier 127). In some aspects, at least the pumps 106, 108, and the piping conduits 110 form a feed system to transport the emulsion 101, demulsifier 127, and other fluids through the HETS 100.

In FIG. 1, the example heating unit 112 is comprised of a fluid-to-fluid heat exchanger 114 (for example, shell and tube, plate and frame, or otherwise). A hot fluid 109 can be heated by one or more solar panel assemblies 116 that convert solar energy 107 into heat energy 129 to raise a temperature of the hot fluid 109 to a preset or desired temperature (or temperature range). Heat energy from the hot fluid 109 is controllably transferred (in other words, at particular or predetermined temperatures) to the mixed emulsion-demulsifier fluid 105 circulated through the heat exchanger 114 to heat the mixed emulsion-demulsifier fluid 105, and an output of cool fluid 111 is returned to receive more heat energy 129. In some aspects, gradually heating the fluid 105 can enhance the breakage of the emulsion 101 (with or without the demulsifier 127), thereby separating it into oil and water more efficiently. In some aspects, when heating is coupled with demulsifier injection, the separation can become more efficient. With this efficiency can come a reduction in an amount of demulsifier 127 needed to break the emulsion 101, while heating is applied and, for example, a maximum steady state temperature is attained in heating unit 112.

A broken emulsion fluid 113 exits the heating unit 112 and enters a separation tank 118 (such as a gravity separation tank) as shown in FIG. 1. In some aspects, such as when no demulsifier 127 is injected, the broken emulsion fluid 113 is the emulsion 101 in a heated state. In some aspects, such as when demulsifier 127 is injected, the broken emulsion fluid 113 is the mixed emulsion-demulsifier fluid 105 in a heated state. In this example implementation, the separation tank 118 operates to separate the broken emulsion fluid 113 into a water phase 121, an emulsion layer 119 (or emulsion phase 119), and an oil phase 117. The oil phase 117 is transported (for example, forcibly or naturally) to an oil tank 120; the water phase 121 is transported (for example, forcibly or naturally) to a water tank 122; and the emulsion layer 119 is transported (for example, forcibly or naturally) to an emulsion tank 124.

In some aspects, an amount of time that the broken emulsion fluid 113 remains in the separation tank 118, while it is separating into the water phase 121, oil phase 117, and emulsion layer 119, is a retention time. In the separation tank 118, the retention time is a total time of emulsion formation (being oil and water) and residence of the emulsion layer 119 in the tank 118 at a designed flow rate. Utilized here in the HETS 100 operating in a batch mode are three main methods to separate oil 117 and water 121 from the emulsion layer 119: chemical, heat, and time. This time is the retention time to aid separation, since when a mixture of oil and water are allowed to stand without agitation, the mixture begins to separate into horizontal sections of oil, free water, and emulsification.

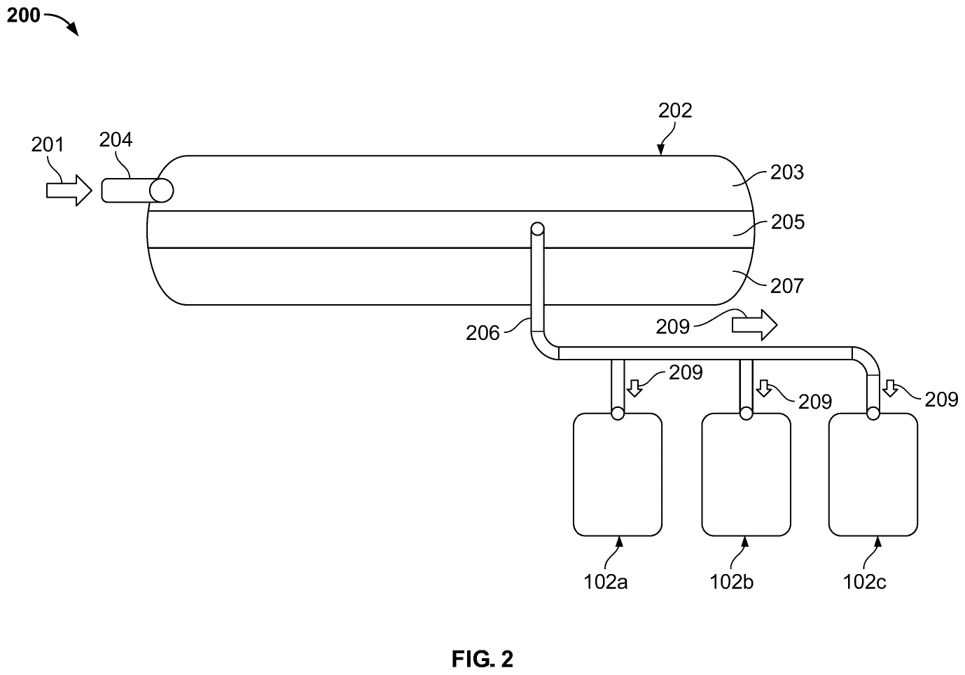

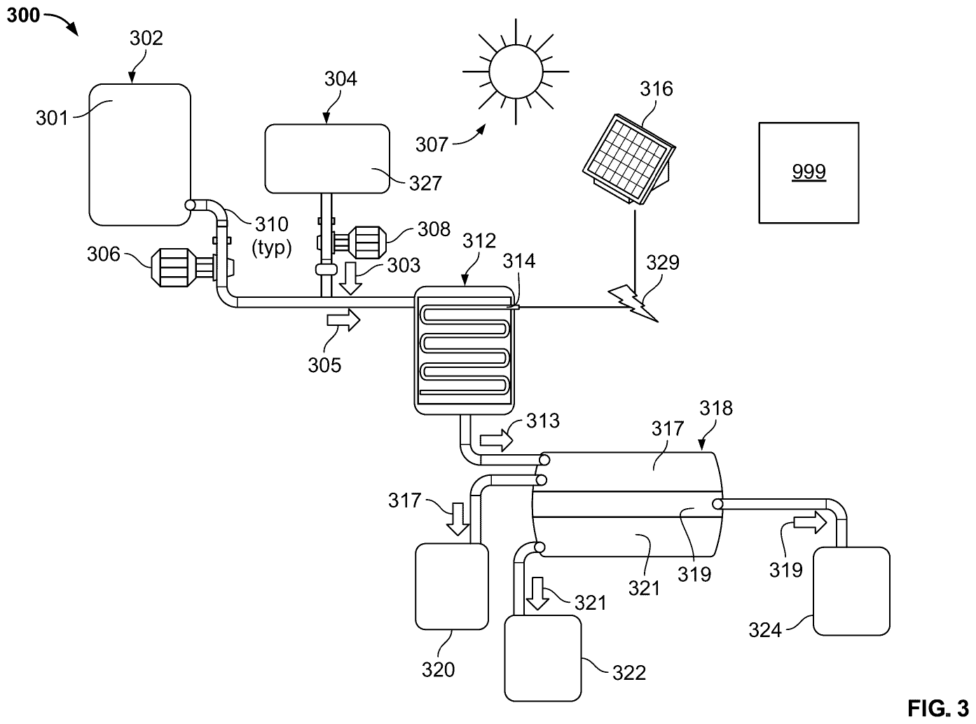

Turning briefly to FIG. 2, this figure shows a schematic diagram of a multiphase fluid production vessel 200 according to the present disclosure. In example aspects, the multiphase fluid production vessel 200 can be used to obtain samples of the emulsion 101 (and 301 shown in FIG. 3). In this example, the multiphase fluid production vessel 200 includes a gravity separation tank 202 that receives a crude oil 201 from a trunkline 204 from oil production wells. The gravity separation tank 202 operates to separate the crude oil 201 into a water phase 207, an oil phase 203, and an emulsion layer 205. An output conduit 206 is fluidly coupled to the gravity separation tank 202 at a height or level of the emulsion layer 205 so that the emulsion layer 205 can be circulated as emulsion fluid 209 into sample tanks 102a, 102b, and 102c. Thus, each sample tank 102a, 102b, and 102c includes a sample of the emulsion 209, which is then tested in the HETS 100 (or a HETS 300 shown in FIG. 3). The sample tanks 102a, 102b, and 102c, themselves, can be used as the emulsion tank 102 shown in FIG. 1 (or an emulsion tank 302 shown in FIG. 3).

By dividing the emulsion 209 into separate sample tanks 102a, 102b, and 102c, batch processes can be performed by the HETS 100 (or HETS 300). Generally, for example, a batch process performed on the emulsion 209 in sample tank 102a can be performed by HETS 100 (or HETS 300) and include no heating and no demulsifier injection. Another batch process performed on the emulsion 209 in sample tank 102b can be performed by HETS 100 (or HETS 300) and include heating but no demulsifier injection. Another batch process performed on the emulsion 209 in sample tank 102c can be performed by HETS 100 (or HETS 300) and include heating and demulsifier injection. The resulting outputs of the different samples as tested by the HETS 100 (or HETS 300)—for example, how much oil phase, water phase, and emulsion layer fluid are collected—can then be quantitatively compared.

Returning to FIG. 1, the process streams of the present disclosure can be flowed using one or more flow control systems 999 (for example, FIGS. 1 and 3) implemented throughout the illustrated hydrocarbon emulsion testing systems shown in the present disclosure. A flow control system 999 can include one or more flow pumps (as shown) to pump the process streams, one or more flow pipes (as shown) through which the process streams are flowed and one or more valves to regulate the flow of streams through the pipes. In the present disclosure, a “pump” or “flow pump” can refer to a liquid pump that forcibly circulates a liquid or mixed phase fluid, a fan that circulates a gas, a compressor that compresses and circulates a fluid, or a turbine that expands and circulates a fluid.

Control system 999 can include one or more monitoring devices. In example implementations, control system 999 can include one or more pressure monitoring devices, temperature monitoring devices, and/or chemical analysis devices to measure constituent species of the example flows shown in FIGS. 1 and 3.

In example implementations, flow control system 999 can include one or more temperature sensors (for example, thermocouples, thermistors, thermometers) and temperature controllers to monitor and control one or more aspects of flow control system 999. In example implementations, flow control system 999 can include one or more power control units, to provide electrical power to components of the illustrated processes.

In example implementations, flow control system 999 can be operated manually. For example, an operator can set a flow rate for each pump and set valve open or close positions to regulate the flow of the process streams through the pipes in flow control system 999. Once the operator has set the flow rates and the valve open or close positions for all flow control systems 999 distributed across the illustrated processes, flow control system 999 can flow the streams under constant flow conditions, for example, constant volumetric rate or other flow conditions. To change the flow conditions, the operator can manually operate control system 999, for example, by changing the pump flow rate or the valve open or close position.

In example implementations, flow control system 999 can be operated automatically. For example, the flow control system 999 can be connected to a computer or a computer-readable medium storing instructions (such as flow control instructions and other instructions) executable by one or more processors to perform operations (such as flow control operations). An operator can set the flow rates and the valve open or close positions for all flow control systems 999 distributed across the illustrated processes using the flow control system 999. In such implementations, the operator can manually change the flow conditions by providing inputs through the flow control system 999. Also, in such implementations, the flow control system 999 can automatically (that is, without manual intervention) control one or more of the flow control systems, for example, using feedback systems connected to flow control system 999. For example, a sensor (such as a pressure sensor, temperature sensor or other sensor) can be connected to a pipe through which a process stream flows. The sensor can monitor and provide a flow condition (such as a pressure, temperature, or other flow condition) of the process stream to flow control system 999. In response to the flow condition exceeding a threshold (such as a threshold pressure value, a threshold temperature value, or other threshold value), control system 999 can automatically perform operations. For example, if the pressure or temperature in the pipe exceeds the threshold pressure value or the threshold temperature value, respectively, flow control system 999 can provide a signal to the pump to decrease a flow rate, a signal to open a valve to relieve the pressure, a signal to shut down process stream flow, or other signals.

An example operation of the HETS 100 can experimentally test multiple emulsion samples. For example, three emulsion samples in sample tanks 102a, 102b, and 102c (of equal volume) are collected from a production vessel, such as at a specific GOSP challenged with the production of tight emulsion. In some aspects, a volume of the emulsion tank 102 is at least twice a volume of each of the water tank 122, the oil tank 120, and the emulsion tank 124. In particular aspects, each of the samples of the emulsion 209 in the tanks 102a, 102b, and 102c is free of a demulsifier prior to testing the samples in the HETS 100.

In a first batch process, a first sample emulsion from sample tank 102a is placed into the emulsion tank 102 (as emulsion 101). The emulsion 101 is circulated (for example, by pump 106) through the HETS 100 without any injection of demulsifier 127 and without any heating applied in the heating unit 112. This emulsion 101 (from the sample in sample tank 102a) should not separate into oil and water; thus, water tank 122 and oil tank 120 should remain empty (or substantially empty) after testing is concluded (after the emulsion 101 travels through the HETS 100 and into the emulsion tank 124.

In a second batch process, a second sample emulsion from sample tank 102b is placed into the emulsion tank 102 (as emulsion 101). The emulsion 101 is circulated (for example, by pump 106) through the HETS 100 without any injection of demulsifier 127. Heat is applied to the emulsion 101 in the heating unit 112. This emulsion 101 (from the sample in sample tank 102b) should separate into oil 117 and water 121 in the separation tank 118. In some aspects, heating is applied incrementally at a specific temperature difference across the heat exchanger 114. The oil tank 120 and the water tank 122 gradually and steadily accumulate oil 117 and water 121, respectively. In some aspects, the flow control system 999 (or operator) can monitor these tanks for additional liquid accumulation.

Once a terminal temperature is reached where no more oil or water are separated, the amount of liquid in each tank 120 and 122 can be precisely measured for each phase accumulated in each tank, separately. In some aspects, the terminal temperature is a temperature in which maximum separation of each phase (oil and water) has been achieved. In other words, the terminal temperature is a maximum temperature that the emulsion layer 119 (that includes oil 117 and water 121) can reach before these phases evaporate or degrade to a less marketable product (in the case of the oil 117).

In a third batch process, a third sample emulsion from sample tank 102c is placed into the emulsion tank 102 (as emulsion 101). The emulsion 101 is circulated (for example, by pump 106) through the HETS 100 with injection of demulsifier 127. Heat is applied to the mixed emulsion-demulsifier fluid 105 in the heating unit 112. This mixed emulsion-demulsifier fluid 105 should separate into oil 117 and water 121 in the separation tank 118. In some aspects, heating is applied incrementally at a specific temperature difference across the heat exchanger 114. The oil tank 120 and the water tank 122 gradually and steadily accumulate oil 117 and water 121, respectively. In some aspects, the flow control system 999 (or operator) can monitor these tanks for additional liquid accumulation.

In some aspects, the demulsifier 127 is injected at an instant in which a terminal temperature for maximum separation of oil and water is reached. At this stage, the demulsifier injection flowrate can be increased incrementally until a maximum amount of liquids (oil and water) are accumulated in the respective oil tank 120, the water tank 122, and the emulsion tank 124.

In some aspects, ascertaining the terminal temperature for a maximum water-oil separation is determined from the separation tank 118. For example, measuring the liquid quantity inside at least one of (or two or three of) the oil tank 120, the water tank 122, and the emulsion tank 124 can show the separation quantity of each phase. The emulsion layer 119 can separate into oil 117 and water 121, with the increase in heating applied incrementally at a specific temperature range. The oil tank 120 can steadily accumulate a precise, measurable separated quantity of oil 117, while the water tank 122 can steadily accumulate a precise, measurable separated quantity of water 121. Once the terminal temperature is reached where no more oil 117 or water 121 can be separated, the amount of liquid in each tank 120 and 122 can be precisely measured for each phase accumulated in each tank, separately. This can provide a threshold of temperature that provides a maximum separation of each phase.

In some aspects, the determination of the terminal temperature can also indicate an amount of demulsifier 127 that should be used to break the emulsion 101. For example, at the terminal temperature, a minimum amount of demulsifier 127 can be used in the process of breaking the emulsion 101 in order to obtain a maximum volume of the oil 117 and the water 121. Thus, the process of breaking the emulsion 101 at the terminal temperature (in other words, incrementally heating the emulsion 101 to the highest temperature possible) can also include injecting the least amount of demulsifier 127 as possible for optimum emulsion breaking.

Further, in some aspects, the processes carried out to test the samples of the emulsion 101 (from sample tanks 102a, 102b, and 102c) can include injecting different demulsifiers 127 for each set of samples. Therefore, different types of demulsifiers 127 can be benchmark tested against each other to determine optimal demulsifier performance in breaking the emulsion 101. For instance, at least the third batch process (and optionally the first batch process and/or the second batch process) can be repeated (one or more times) with different demulsifiers 127. At each repetition, an amount of demulsifier 127 can be measured or otherwise determined to make a comparison between demulsifiers based on the amount used, as well as the volumes of the oil phase 117, the water phase 121, and the emulsion phase 119 collected in the respective tanks 120, 122, and 124.

FIG. 3 is a schematic diagram of another example implementation of a hydrocarbon emulsion testing system according to the present disclosure. In some aspects, the HETS 300 can be a stand along system as part of an independent laboratory remote from a GOSP. Alternatively, the HETS 300 can be implemented as part of, and adjacent or within, a GOSP.

In this example, the HETS 300 includes an emulsion tank 302 in which an emulsion 301 (for example, a multiphase fluid that includes oil, water, emulsion, gas or a combination thereof) is enclosed. The emulsion tank 302 is fluidly coupled within the HETS 300, as are the other described components, with piping conduits 310 (for example, metallic, non-metallic). As shown here, a pump 306 is fluidly coupled within the piping conduits 310 to circulate the emulsion 301 within the HETS 300 (or at least a portion thereof).

In this example, a demulsifier tank 304 that encloses a demulsifier 327 is fluidly coupled within the HETS 300 through piping conduits 310. The demulsifier 327, in some aspects, is a chemical compound formulated to break emulsion layers within the emulsion 301 (and that typically form inside of oil and water production vessels). Demulsifier 327 can be injected into the HETS 300 and in contact with the emulsion 301 through operation of pump 308. Thus, in some example operations (as described in more detail herein), a mixed emulsion-demulsifier fluid 305 is circulated to a heating unit 312. In some example operations (as described in more detail herein), however, the fluid 305 is the emulsion 301 (in other words, with no demulsifier 327). In some aspects, at least the pumps 306, 308, and the piping conduits 310 form a feed system to transport the emulsion 301, demulsifier 327, and other fluids through the HETS 300.

In FIG. 3, the example heating unit 312 is comprised of an electric heater 314 in which electric heat 329 is generated by one or more solar panel assemblies 316 that convert solar energy 307 into the electric heat 329. The electric heat 329 is controllably transferred (in other words, at particular or predetermined temperatures) to the mixed emulsion-demulsifier fluid 305 circulated through the electric heater 314 to heat the mixed emulsion-demulsifier fluid 305. In some aspects, gradually heating the fluid 305 can enhance the breakage of the emulsion 301 (with or without the demulsifier 327), thereby separating it into oil and water more efficiently. In some aspects, when heating is coupled with demulsifier injection, the separation can become more efficient. With this efficiency can come a reduction in an amount of demulsifier 327 needed to break the emulsion 301, while heating is applied and, for example, a maximum steady state temperature is attained in heating unit 312.

Other heating sources can be used for heating unit 312 (or heating unit 112). For example, there are often energy sources available for heating in the HETS 300, which can include any available external source such as heated feed water available at a building, auxiliary steam lines, auxiliary electric power available at a GOSP.

A broken emulsion fluid 313 exits the heating unit 312 and enters a separation tank 318 (such as a gravity separation tank) as shown in FIG. 3. In some aspects, such as when no demulsifier 327 is injected, the broken emulsion fluid 313 is the emulsion 301 in a heated state. In some aspects, such as when demulsifier 327 is injected, the broken emulsion fluid 313 is the mixed emulsion-demulsifier fluid 305 in a heated state. In this example implementation, the separation tank 318 operates to separate the broken emulsion fluid 313 into a water phase 321, an emulsion layer 319 (or an emulsion phase 119), and an oil phase 317. The oil phase 317 is transported (for example, forcibly or naturally) to an oil tank 320; the water phase 321 is transported (for example, forcibly or naturally) to a water tank 322; and the emulsion layer 319 is transported (for example, forcibly or naturally) to an emulsion tank 324.

In some aspects, an amount of time that the broken emulsion fluid 313 remains in the separation tank 318, while it is separating into the water phase 321, oil phase 317, and emulsion layer 319, is a retention time. In the separation tank 318, the retention time is a total time of emulsion formation (being oil and water) and residence of the emulsion layer 319 in the tank 318 at a designed flow rate. Utilized here in the HETS 300 operating in a batch mode are three main methods to separate oil 317 and water 321 from the emulsion layer 319: chemical, heat, and time. This time is the retention time to aid separation, since when a mixture of oil and water are allowed to stand without agitation, the mixture begins to separate into horizontal sections of oil, free water, and emulsification.

An example operation of the HETS 300 can experimentally test multiple emulsion samples. For example, three emulsion samples in sample tanks 102a, 102b, and 102c (of equal volume) are collected from a production vessel, such as at a specific GOSP challenged with the production of tight emulsion. In some aspects, a volume of the emulsion tank 302 is at least twice a volume of each of the water tank 322, the oil tank 320, and the emulsion tank 324. In particular aspects, each of the samples of the emulsion 209 in the tanks 102a, 102b, and 102c is free of a demulsifier prior to testing the samples in the HETS 300.

In a first batch process, a first sample emulsion from sample tank 102a is placed into the emulsion tank 302 (as emulsion 301). The emulsion 301 is circulated (for example, by pump 306) through the HETS 300 without any injection of demulsifier 327 and without any heating applied in the heating unit 312. This emulsion 301 (from the sample in sample tank 102a) should not separate into oil and water; thus, water tank 322 and oil tank 320 should remain empty (or substantially empty) after testing is concluded (after the emulsion 301 travels through the HETS 300 and into the emulsion tank 324.

In a second batch process, a second sample emulsion from sample tank 102b is placed into the emulsion tank 302 (as emulsion 301). The emulsion 301 is circulated (for example, by pump 306) through the HETS 300 without any injection of demulsifier 327. Heat is applied to the emulsion 301 in the heating unit 312. This emulsion 301 (from the sample in sample tank 102b) should separate into oil 317 and water 321 in the separation tank 318. In some aspects, heating is applied incrementally at a specific temperature difference across the electric heater 314. The oil tank 320 and the water tank 322 gradually and steadily accumulate oil 317 and water 321, respectively. In some aspects, the flow control system 999 (or operator) can monitor these tanks for additional liquid accumulation.

Once a terminal temperature is reached where no more oil or water are separated, the amount of liquid in each tank 320 and 322 can be precisely measured for each phase accumulated in each tank, separately. In some aspects, the terminal temperature is a temperature in which maximum separation of each phase (oil and water) has been achieved. In other words, the terminal temperature is a maximum temperature that the emulsion layer 319 (that includes oil 317 and water 321) can reach before these phases evaporate or degrade to a less marketable product (in the case of the oil 317).

In a third batch process, a third sample emulsion from sample tank 102c is placed into the emulsion tank 302 (as emulsion 301). The emulsion 301 is circulated (for example, by pump 306) through the HETS 300 with injection of demulsifier 327. Heat is applied to the mixed emulsion-demulsifier fluid 305 in the heating unit 312. This mixed emulsion-demulsifier fluid 305 should separate into oil 317 and water 321 in the separation tank 318. In some aspects, heating is applied incrementally at a specific temperature difference across the electric heater 314. The oil tank 320 and the water tank 322 gradually and steadily accumulate oil 317 and water 321, respectively. In some aspects, the flow control system 999 (or operator) can monitor these tanks for additional liquid accumulation.

In some aspects, the demulsifier 327 is injected at an instant in which a terminal temperature for maximum separation of oil and water is reached. At this stage, the demulsifier injection flowrate can be increased incrementally until a maximum amount of liquids (oil and water) are accumulated in the respective oil tank 320, the water tank 322, and the emulsion tank 324.

In some aspects, ascertaining the terminal temperature for a maximum water-oil separation is determined from the separation tank 318. For example, measuring the liquid quantity inside at least one of (or two or three of) the oil tank 320, the water tank 322, and the emulsion tank 324 can show the separation quantity of each phase. The emulsion layer 319 can separate into oil 317 and water 321, with the increase in heating applied incrementally at a specific temperature range. The oil tank 320 can steadily accumulate a precise, measurable separated quantity of oil 317, while the water tank 322 can steadily accumulate a precise, measurable separated quantity of water 321. Once the terminal temperature is reached where no more oil 317 or water 321 can be separated, the amount of liquid in each tank 320 and 322 can be precisely measured for each phase accumulated in each tank, separately. This can provide a threshold of temperature that provides a maximum separation of each phase.

In some aspects, the determination of the terminal temperature can also indicate an amount of demulsifier 327 that should be used to break the emulsion 301. For example, at the terminal temperature, a minimum amount of demulsifier 327 can be used in the process of breaking the emulsion 301 in order to obtain a maximum volume of the oil 317 and the water 321. Thus, the process of breaking the emulsion 301 at the terminal temperature (in other words, incrementally heating the emulsion 301 to the highest temperature possible) can also include injecting the least amount of demulsifier 327 as possible for optimum emulsion breaking.

Further, in some aspects, the processes carried out to test the samples of the emulsion 301 (from sample tanks 102a, 102b, and 102c) can include injecting different demulsifiers 327 for each set of samples. Therefore, different types of demulsifiers 327 can be benchmark tested against each other to determine optimal demulsifier performance in breaking the emulsion 301. For instance, at least the third batch process (and optionally the first batch process and/or the second batch process) can be repeated (one or more times) with different demulsifiers 327. At each repetition, an amount of demulsifier 327 can be measured or otherwise determined to make a comparison between demulsifiers based on the amount used, as well as the volumes of the oil phase 317, the water phase 321, and the emulsion phase 319 collected in the respective tanks 320, 322, and 324.

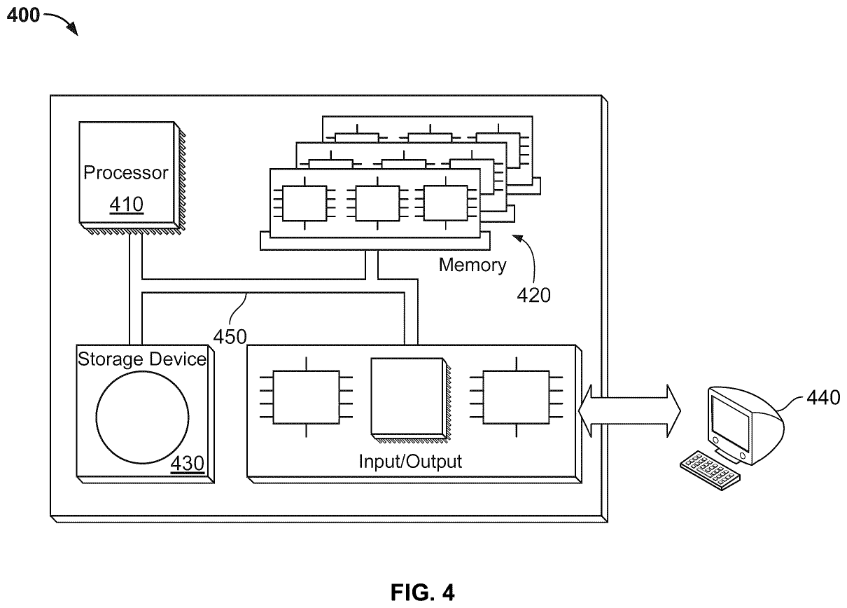

FIG. 4 is a schematic illustration of an example controller (or control system) 400 for a hydrocarbon emulsion testing system according to the present disclosure. For example, the controller 400 may include or be part of a control system 999 shown as part of hydrocarbon emulsion testing systems 100 or 300 according to the present disclosure. The controller 400 is intended to include various forms of digital computers, such as printed circuit boards (PCB), processors, digital circuitry, or otherwise parts of a hydrocarbon emulsion testing system. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 400 includes a processor 410, a memory 420, a storage device 430, and an input/output device 440. Each of the components 410, 420, 430, and 440 are interconnected using a system bus 450. The processor 410 is capable of processing instructions for execution within the controller 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the controller 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the controller 400. In one implementation, the storage device 430 is a computer-readable medium. In various different implementations, the storage device 430 may be a floppy disk device, a hard disk device, an optical disk device, or a tape device.

The input/output device 440 provides input/output operations for the controller 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat panel displays and other appropriate mechanisms.

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

What is claimed is:

1. A hydrocarbon emulsion testing process, comprising:
feeding a first sample of a hydrocarbon emulsion at a first temperature into a hydrocarbon emulsion testing system (HETS), the hydrocarbon emulsion comprising an oil phase, a water phase, and an emulsion phase;
separating, in a separation tank of the HETS, the first sample of the hydrocarbon emulsion at the first temperature into a first volume of the oil phase, a first volume of the water phase, and a first volume of an emulsion phase;
feeding a second sample of the hydrocarbon emulsion at the first temperature into the HETS;
heating, in a heating unit of the HETS, the second sample of the hydrocarbon emulsion from the first temperature to a second temperature;
separating, in the separation tank, the second sample of the hydrocarbon emulsion at the second temperature into a second volume of the oil phase, a second volume of the water phase, and a second volume of the emulsion phase;
feeding a third sample of the hydrocarbon emulsion at the first temperature into the HETS;
injecting, from a demulsifier tank of the HETS, a demulsifier into the third sample of the hydrocarbon emulsion;
heating, in the heating unit, the third sample of the hydrocarbon emulsion from the first temperature to a third temperature;
separating, in the separation tank, the third sample of the hydrocarbon emulsion at the third temperature into a third volume of the oil phase, a third volume of the water phase, and a third volume of the emulsion phase;
comparing the first, second, and third volumes of at least one of the oil phase, the water phase, or the emulsion phase; and
based on the comparison, determining a terminal temperature of the HETS.

2. The hydrocarbon emulsion testing process of claim 1, comprising, based on at least one of the comparison or the determined terminal temperature, determining an optimum volume of the injected demulsifier.

3. The hydrocarbon emulsion testing process of claim 1, wherein comparing the first, second, and third volumes of at least one of the oil phase, the water phase, or the emulsion phase comprises:
comparing the first, second, and third volumes of the oil phase; and
comparing the first, second, and third volumes of the water phase.

4. The hydrocarbon emulsion testing process of claim 1, wherein determining the terminal temperature of the HETS comprises at least one of:
determining that the third volume of the oil phase is greater than the first and second volumes of the oil phase and is a maximum volume of the oil phase within the hydrocarbon emulsion; or
determining that the third volume of the water phase is greater than the first and second volumes of the water phase and is a maximum volume of the water phase within the hydrocarbon emulsion.

5. The hydrocarbon emulsion testing process of claim 4, wherein determining the terminal temperature of the HETS comprises:
determining that the third volume of the oil phase is greater than the first and second volumes of the oil phase and is a maximum volume of the oil phase within the hydrocarbon emulsion; and
determining that the third volume of the water phase is greater than the first and second volumes of the water phase and is a maximum volume of the water phase within the hydrocarbon emulsion.

6. The hydrocarbon emulsion testing process of claim 1, wherein heating the second sample of the hydrocarbon emulsion and the third sample of the hydrocarbon emulsion in the heating unit of the HETS comprises:
heating, in a fluid-to-fluid heat exchanger of the heating unit, the second sample of the hydrocarbon emulsion with a heating fluid that comprises heat energy from solar energy; and
heating, in the fluid-to-fluid heat exchanger, the third sample of the hydrocarbon emulsion with the heating fluid that comprises heat energy from solar energy.

7. The hydrocarbon emulsion testing process of claim 1, wherein heating the second sample of the hydrocarbon emulsion and the third sample of the hydrocarbon emulsion in the heating unit of the HETS comprises:
heating, in an electric heater of the heating unit, the second sample of the hydrocarbon emulsion with heat energy from solar energy; and
heating, in the electric heater, the third sample of the hydrocarbon emulsion with heat energy from solar energy.

8. The hydrocarbon emulsion testing process of claim 1, wherein each of the first, second, and third samples of the hydrocarbon emulsion is exclusive of demulsifier prior to the feeding into the HETS.

9. The hydrocarbon emulsion testing process of claim 1, wherein the demulsifier comprises a first demulsifier, and the process comprises:
feeding a fourth sample of the hydrocarbon emulsion at the first temperature into the HETS;
injecting, from the demulsifier tank, a second demulsifier into the fourth sample of the hydrocarbon emulsion, the second demulsifier different than the first demulsifier;
heating, in the heating unit, the fourth sample of the hydrocarbon emulsion from the first temperature to the third temperature;
separating, in the separation tank, the fourth sample of the hydrocarbon emulsion at the third temperature into a fourth volume of the oil phase, a fourth volume of the water phase, and a fourth volume of the emulsion phase;
comparing the third and fourth volumes of at least one of the oil phase, the water phase, or the emulsion phase; and
based on the comparison, selecting one of the first or second demulsifiers as an optimum demulsifier.

10. The hydrocarbon emulsion testing process of claim 1, wherein the terminal temperature is the third temperature.

11. The hydrocarbon emulsion testing process of claim 1, wherein heating the third sample of the hydrocarbon emulsion from the first temperature to the third temperature comprises:
heating, in the heating unit, the third sample of the hydrocarbon emulsion that includes the demulsifier from the first temperature to the third temperature.

12. A hydrocarbon emulsion testing system, comprising:
a fluid feeding system comprising at least one pump and a fluid piping network, the fluid feeding system configured to:
feed a first sample of a hydrocarbon emulsion at a first temperature into the fluid piping network, the hydrocarbon emulsion comprising an oil phase, a water phase, and an emulsion phase;
feed a second sample of the hydrocarbon emulsion at the first temperature into the fluid piping network; and
feed a third sample of the hydrocarbon emulsion at the first temperature into the fluid piping network;
a demulsifier tank fluidly coupled within the fluid piping network and configured to inject a demulsifier into the third sample of the hydrocarbon emulsion;
a heating unit thermally coupled to the fluid piping network and configured to:
heat the second sample of the hydrocarbon emulsion from the first temperature to a second temperature; and
heat the third sample of the hydrocarbon emulsion from the first temperature to a third temperature;
a separation tank fluidly coupled within the fluid piping network and configured to:
separate the first sample of the hydrocarbon emulsion at the first temperature into a first volume of the oil phase, a first volume of the water phase, and a first volume of an emulsion phase;
separate the second sample of the hydrocarbon emulsion at the second temperature into a second volume of the oil phase, a second volume of the water phase, and a second volume of the emulsion phase; and
separate the third sample of the hydrocarbon emulsion at the third temperature into a third volume of the oil phase, a third volume of the water phase, and a third volume of the emulsion phase; and
a control system configured to perform operations comprising:
comparing the first, second, and third volumes of at least one of the oil phase, the water phase, or the emulsion phase; and
based on the comparison, determining a terminal temperature.

13. The hydrocarbon emulsion testing system of claim 12, wherein the operations comprise determining an optimum volume of the injected demulsifier based on at least one of the comparison or the determined terminal temperature.

14. The hydrocarbon emulsion testing system of claim 12, wherein the operation of comparing the first, second, and third volumes of at least one of the oil phase, the water phase, or the emulsion phase comprises:
comparing the first, second, and third volumes of the oil phase; and
comparing the first, second, and third volumes of the water phase.

15. The hydrocarbon emulsion testing system of claim 12, wherein the operation of determining the terminal temperature of the HETS comprises at least one of:
determining that the third volume of the oil phase is greater than the first and second volumes of the oil phase and is a maximum volume of the oil phase within the hydrocarbon emulsion; or
determining that the third volume of the water phase is greater than the first and second volumes of the water phase and is a maximum volume of the water phase within the hydrocarbon emulsion.

16. The hydrocarbon emulsion testing system of claim 15, wherein the operation of determining the terminal temperature of the HETS comprises:
determining that the third volume of the oil phase is greater than the first and second volumes of the oil phase and is a maximum volume of the oil phase within the hydrocarbon emulsion; and
determining that the third volume of the water phase is greater than the first and second volumes of the water phase and is a maximum volume of the water phase within the hydrocarbon emulsion.

17. The hydrocarbon emulsion testing system of claim 12, wherein the heating unit comprises a fluid-to-fluid heat exchanger configured to:
heat the second sample of the hydrocarbon emulsion with a heating fluid that comprises heat energy from solar energy; and
heat the third sample of the hydrocarbon emulsion with the heating fluid that comprises heat energy from solar energy.

18. The hydrocarbon emulsion testing system of claim 12, wherein the heating unit comprises an electric heater configured to:
heat, the second sample of the hydrocarbon emulsion with heat energy from solar energy; and
heat the third sample of the hydrocarbon emulsion with heat energy from solar energy.

19. The hydrocarbon emulsion testing system of claim 12, wherein each of the first, second, and third samples of the hydrocarbon emulsion is exclusive of demulsifier prior to the feeding into the HETS.

20. The hydrocarbon emulsion testing system of claim 12, wherein the demulsifier comprises a first demulsifier,
the fluid feeding system is configured to feed a fourth sample of the hydrocarbon emulsion at the first temperature into the fluid piping network,
the demulsifier tank is configured to inject a second demulsifier into the fourth sample of the hydrocarbon emulsion,
the heating unit is configured to heat the fourth sample of the hydrocarbon emulsion from the first temperature to a fourth temperature,
the separation tank is configured to separate the fourth sample of the hydrocarbon emulsion at the fourth temperature into a fourth volume of the oil phase, a fourth volume of the water phase, and a fourth volume of an emulsion phase, and
the operations comprise:
comparing the third and fourth volumes of at least one of the oil phase, the water phase, or the emulsion phase; and
based on the comparison, selecting one of the first or second demulsifiers as an optimum demulsifier.

21. The hydrocarbon emulsion testing system of claim 12, wherein the terminal temperature is the third temperature.

22. The hydrocarbon emulsion testing system of claim 12, wherein the heating unit is positioned downstream of the demulsifier tank in the fluid piping network, and the heating unit is configured to heat a mixture of the third sample of the hydrocarbon emulsion and the demulsifier from the first temperature to the third temperature.
